(19) United States
(12) Patent Application Publication
(10) Pub. No.: US 2026/0298885 A1
(43) Pub. Date: Oct. 1, 2026

(54) Quantitative Analysis of Polysorbate Oxidation

(71) Applicant: Regeneron Pharmaceuticals, Inc., Tarrytown, NY (US)

(72) Inventors: Hui XIAO, Ridgewood, NJ (US); Sisi ZHANG, White Plains, NY (US)

(21) Appl. No.: 19/529,541

(22) Filed: Feb. 4, 2026

Related U.S. Application Data
(60) Provisional application No. 63/754,738, filed on Feb. 6, 2025.

(51) Int. Cl. G01N 30/06; G01N 30/72; G01N 33/15

(52) U.S. Cl. CPC G01N 30/06; G01N 30/7233; G01N 33/15; G01N 2030/067

(57) ABSTRACT

Methods for assessing oxidation of a polysorbate in a formulation can include determining a concentration of one or more oxidation markers of the polysorbate in a formulation that at least initially included the polysorbate. The one or more oxidation markers can include or can be a compound having carboxylic acid and/or an aldehyde.

### CROSS-REFERENCE TO RELATED APPLICATIONS

This application claims priority to U.S. Provisional Application No. 63/754,738 filed Feb. 6, 2025, the entire disclosure of which is hereby incorporated by reference herein.

### TECHNICAL FIELD

The present application relates to methods for assessing oxidation of polysorbate in a formulation.

### BACKGROUND

Polysorbates (PS) are commonly used surfactants in biopharmaceuticals to protect proteins against surface adsorption and interfacial stresses. PS can be degraded by two mechanisms: hydrolysis and oxidation. The stability of PS in formulations, e.g., drug product formulations, is a concern because its degradation can lead to instability of the protein in the formulation and due to the formation of particles in the formulation. Hence, there is a continuing need for methods for quantifying PS oxidation in formulations.

### SUMMARY OF THE DISCLOSURE

The present disclosure is directed to assessing oxidation of a polysorbate in a formulation that included the polysorbate, e.g., polysorbate 80, by determining a concentration of an oxidation marker, e.g., a carboxylic acid and/or an aldehyde such as octanoic acid.

In one aspect, a method for assessing oxidation of a polysorbate in a formulation, includes determining a concentration of one or more oxidation markers of the polysorbate in a formulation that at least initially included the polysorbate, wherein the one or more oxidation markers comprise a carboxylic acid and/or an aldehyde.

In some embodiments, the polysorbate includes polysorbate 80 (PS80). The one or more oxidation markers can include acetic acid, formic acid, octanoic acid, or nonanoic acid. The formulation can be substantially free of protein.

In some embodiments, the method further includes storing the formulation under storage conditions for a period of time prior to determining the concentration of one or more oxidation markers.

The period of time can be up to about 61 months. The storage conditions can include one or more stress conditions including thermal stress, oxidative stress, or a combination thereof. The storage conditions can include storage at a constant temperature, wherein the constant temperature is between about 2° C. and about 40° C., and the period of time is up to about 61 months.

In some embodiments, determining the concentration of the one or more oxidation markers includes: (a) derivatizing the one or more oxidation markers; and (b) detecting one or more of the derivatized oxidation markers by a liquid chromatography-mass spectrometry (LCMS) method.

In another aspect, a method for assessing oxidation of polysorbate 80 (PS80) in a formulation includes determining a concentration of octanoic acid in the formulation at a first time point.

The method can further include determining a concentration of octanoic acid in the formulation at a second time point later than the first time point. Determining the concentration of octanoic acid in the formulation can include: (a) derivatizing the octanoic acid; and (b) detecting the derivatized octanoic acid by a liquid chromatography-mass spectrometry (LCMS) method. Derivatizing the octanoic acid can include derivatizing with 2-hydrazinoquinoline. The LCMS method can include multiple reaction monitoring (LC-MRM). In some embodiments, the method further includes determining a concentration of octanoic acid in the formulation at a second time point later than the first time point.

Additional advantages of the present disclosure will become readily apparent to those skilled in the art from the following detailed description, wherein only certain embodiments are shown and described, simply by way of illustration of carrying out certain subject matter. As will be realized, the invention is capable of other and different embodiments, and its several details are capable of modifications in various respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent similar elements throughout and wherein:

FIG. 1A, FIG. 1B, FIG. 1C and FIG. 1D show LC-CAD data for PS80 in different stages of oxidation. FIG. 1A, no oxidation: PS80 species remained intact, and no oxidized species can be observed. FIG. 1B, Mild oxidation: initial oxidative PS80 species can be detected, eluting from 22.5 to 25 mins (peak 9 and peak 10) and 29 to 33 mins (peak 11 and peak 12). Slight decrease of PS80 species can be detected. FIG. 1C, Medium oxidation: a variety of PS80 oxidized species can be detected, eluting from 15 to 25 mins (peak 13) and 29 to 33 mins. Obvious decrease of PS80 species can be observed. FIG. 1D, High oxidation: Both PS80 species and PS80 oxidized species had been degraded. A more detailed oxidation stage had been defined in each figure from stage 0 to 7. The identification of the major species in each peak was confirmed by LC-MS as 1. POE sorbitan C18: 2; 2. POE sorbitan C18: 1; 3. POE isosorbide C18: 1; 4. POE sorbitan C18: 1/C18: 2 diester; 5. POE sorbitan di-C18: 1; 6. POE isosorbide di-C18: 1; 7. POE sorbitan tri-oleate; 8. POE sorbitan tetra-oleate. 9. POE sorbitan epoxy-stearate, POE sorbitan hydroperoxyl-oleate, POE sorbitan keto-oleate, etc. 10. POE isosorbide keto-linoleic acid, POE isosorbide epoxy-stearic acid, etc. 11. POE sorbitan 9-oxononanoic acid diester. 12. POE sorbitan, oleic acid/2-decenedioic acid diester; 13. POE oxo-nonanoate.

FIGS. 1E-1 to 1E-2 show the EIC profile of POE sorbitan mono-oleate at different PS80 oxidation stages, eluting between 24-25 min.

FIGS. 1F-1 to 1F-3 show time courses for potential PS80 oxidation products. 0.2% (w/w) polysorbate 80 solution had been incubated under thermal stress condition at 37° C. for 7, 14, 21, 28 and 42 days. Multiple PS80 oxidative products including different ketones, aldehydes and acids were been detected and quantitated.

FIG. 2A and FIG. 2B show the time course of potential markers of PS80 oxidation status. PS80 samples were stressed under different time, temperature, and catalyst level, octanoic acid level was presented as relative abundance by normalizing its signal with internal standard signal. 0.2% (w/w) polysorbate 80 solution in 10 mM histidine, 10% sucrose, pH 6.0 had been incubated at 5° C., 25° C. and 40° C. for up to 6 months. FIG. 2A, Acid markers. FIG. 2B, Aldehyde markers.

FIG. 3A, FIG. 3B, FIG. 3C and FIG. 3D illustrate octanoic acid concentration under different oxidation conditions. PS80 samples were stressed under multiple time points, temperature, and catalyst level, the octanoic acid concentration was quantified. FIG. 3A, 0.1% (w/w) polysorbate 80 solution had been incubated under thermal stress condition at 37° C. for 7, 14, 21, 28 and 42 days. FIG. 3B, 0.1% (w/w) polysorbate 80 solution in 10 mM histidine, 10% sucrose, pH 6.0 had been incubated at 5° C., 25° C. and 40° C. for up to 6 months. In FIGS. 3C-3D, 0 ppb, 1 ppb, 3 ppb, 10 ppb and 30 ppb of iron had been spiked into 0.1% (w/w) polysorbate 80, 10 mM histidine, 10% sucrose, pH 6.0 solution to generate F1, F2, F3, F4 and F5 samples, respectively. FIG. 3C, samples had been incubated at 25° C. for 6 months. FIG. 3D, samples had been incubated at 40° C. for 0.5 months.

FIG. 4A, FIG. 4B, FIG. 4C and FIG. 4D show quantitation of octanoic acid by LC-MRM using precursor ion m/z 286.19 (z=1) and product ion m/z 160.09 (z=1). FIG. 4A, blank.

FIG. 4B, reagent blank. FIG. 4C, lower limit of quantitation (LLOQ). FIG. 4D, calibration curve plots of octanoic acid (50 ng/mL to 50 μg/mL). PAR was calculated by dividing the peak areas of octanoic acid by the peak areas of d4-acetic acid.

FIG. 5A and FIG. 5B show overlaid LC-CAD profile for PS-80 at different oxidation stages.

FIG. 6A and FIG. 6B show octanoic acid concentration at different PS80 oxidation stages. FIG. 6A: 0.1% (w/w) PS80 in the formulation. FIG. 6B: 0.1% (w/w) polysorbate 80 solution had been incubated with 0.02% H₂O₂ and 200 ppb iron spiked in at 5° C. for up to 19 days. Octanoic acid concentration was calculated by average multiple data points under different stressed conditions.

FIG. 7A and FIG. 7B show percentage of PS80 corresponding to oxidation stages.

FIG. 7A: 0.05% (w/w), 0.1% (w/w) 0.15% (w/w) and 0.2% (w/w) PS80 solution had been exposed under oxidative stress with 0.02% H₂O₂ and 200 ppb iron spiked in and incubated at 37° C. for up to 8 days. FIG. 7B: Octanoic acid standard was under oxidative stress with 0.02% H₂O₂ and 200 ppb FeCl₃ spiked in and was incubated at 37° C. for up to 19 days. Octanoic acid concentration had been quantitated at different time points representing different PS80 oxidation stage.

### DETAILED DESCRIPTION OF THE DISCLOSURE

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Polysorbates (PS) are fatty acid esters of polyethoxylated sorbitan (polyoxyethylene sorbitan esters). The polyoxyethylene serves as the hydrophilic head group and the fatty acid as the lipophilic tail. The effectiveness as a surfactant of the polysorbate depends upon both groups being present in a single molecule.

Polysorbate 20, polysorbate 40, polysorbate 60, and polysorbate 80 are widely employed in the pharmaceutical, cosmetic, and food industries as stabilizers and emulsifiers. Polysorbate 20 mostly comprises the monolaurate ester of polyoxyethylene (20) sorbitan. Polysorbate 40 mostly comprises the monopalmitate ester of polyoxyethylene (20) sorbitan. Polysorbate 60 mostly comprises the monostearate ester of polyoxyethylene (20) sorbitan. Polysorbate 80 mostly comprises the monooleate ester of polyoxyethylene (20) sorbitan.

The quality of commercial grades of polysorbates varies from vendor to vendor. Polysorbates therefore are often mixtures of various chemical entities, consisting mostly of polyoxyethylene (20) sorbitan monoesters (as described above) with, in some cases, isosorbide ester contaminants. The head group (e.g., polyoxyethylene (20) sorbitan) comprises a sorbitan (a mixture of dehydrated sorbitols, including 1,4-anhydrosorbitol, 1,5-anhydrosorbitol, and 1,4,3,6-dianhydrosorbitol) substituted at three of its alcohol groups to form ether bonds with three polyoxyethylene groups. The fourth alcohol group is substituted with a fatty acid to form a fatty acid ester.

In some commercially available batches of polysorbates, the polysorbate contains isosorbide monoesters. Isosorbide is a heterocyclic derivative of glucose, also prepared by the dehydration of sorbitol. It is a diol, i.e., having two alcohol groups that can take part in the formation of one or two ester bonds. Thus, for example, some lots of polysorbate 20 can contain significant amounts of isosorbide laurate mono- and di-esters.

In addition to head group variation, preparations of polysorbates contain variable amounts of other fatty acid esters. For example, an analysis of one particular source of polysorbate 20 revealed<10% caprylic acid, <10% capric acid, 40-60% lauric acid, 14-25% myristic acid, 7-15% palmitic acid, <11% oleic acid, <7% stearic acid, and <3% linoleic acid. An analysis of a polysorbate 80 batch revealed<5% myristic acid, <16% palmitic acid, >58% oleic acid, <6% stearic acid, and <18% linoleic acid.

The term “fatty acid” or “fatty acid chain” means a carboxylic acid having an aliphatic tail. An aliphatic tail is a hydrocarbon chain comprising carbon and hydrogen, and in some cases, oxygen, sulfur, nitrogen and/or chlorine substitutions. Aliphatic tails can be saturated (as in saturated fatty acids), which means that all carbon-carbon bonds are single bonds (i.e., alkanes). Aliphatic tails can be unsaturated (as in unsaturated fatty acids), wherein one or more carbon-carbon bonds are double bonds (alkenes), or triple bonds (alkynes).

Fatty acids are generally designated as short-chain fatty acids, which have fewer than six carbons in their aliphatic tails, medium-chain fatty acids having six to twelve carbons, long-chain fatty acids having thirteen to twenty one carbons, and very long chain fatty acids having aliphatic tails of twenty two carbons and longer. As mentioned above, fatty acids are also categorized according to their degree of saturation. Common fatty acids include caprylic acid (8 carbons: 0 double bonds; 8:0), capric acid (10:0), lauric acid (12:0), myristic acid (14:0), myristoleic acid (14:1), palmitic acid (16:0), palmitoleic acid (16:1), sapienic acid (16:1), stearic acid (18:0), oleic acid (18:1), elaidic acid (18:1), vaccenic acid (18:1), linoleic acid (18:2), linelaedic acid (18:2), alpha-linolenic acid (18:3), arachidic acid (20:0), arachidonic acid (20:4), eicosapentaenoic acid (20:5), behenic acid (22:0), erucic acid (22:1), docosahexaenoic acid (22:6), lignoceric acid (24:0), and cerotic acid (26:0).

The term “protein” means any amino acid polymer having more than about 20 amino acids covalently linked via amide bonds. Proteins contain one or more amino acid polymer chains, generally known in the art as “polypeptides”. Thus, a polypeptide may be a protein, and a protein may contain multiple polypeptides to form a single functioning biomolecule of a single conformation. Disulfide bridges (between cysteine residues to form cystine) may be present in some proteins. These covalent links may be within a single polypeptide chain, or between two individual polypeptide chains.

In addition to disulfide bond formation, proteins may be subject to other post-translational modifications. Those modifications include lipidation (e.g., myristoylation, palmitoylation, farnesoylation, geranylgeranylation, and glycosylphosphatidylinositol (GPI) anchor formation), alkylation (e.g., methylation), acylation, amidation, glycosylation (e.g., addition of glycosyl groups at arginine, asparagine, cysteine, hydroxylysine, serine, threonine, tyrosine, and/or tryptophan), and phosphorylation (i.e., the addition of a phosphate group to serine, threonine, tyrosine, and/or histidine).

A protein of the present disclosure includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, cytokines, chemokines, peptide hormones, and the like.

The stability of PS in formulations, e.g., drug product formulations, is a concern due to the formation of visible and subvisible particles as well as decreased protection for active ingredients such as proteins.

PS can be degraded by hydrolysis and/or oxidation. In hydrolysis, the fatty acid ester bond is cleaved. Hydrolysis can be promoted by e.g., trace levels of host cell proteins such as lipase or esterase present in protein drug products. Hydrolytic degradation can result in particle formation in drug products. In oxidation, the POE chain can be cleaved at the ethylene oxide subunits; and/or the unsaturated alkyl chain of the fatty acid tail (derived from, e.g., oleic acid or linolenic acid), can be cleaved. See, for example, Liu H, et al. J Pharm Sci. 2022; 111 (2): 323-34.

The initial products of oxidative degradation can undergo further oxidation, forming a wide range of small organic species. Such species include, but are not limited to, peroxides, alkanes, aldehydes, ketones, and acids. Polysorbate oxidation results in a variety of primary and secondary oxidative products. Primary oxidative products include POE sorbitan hydroperoxyl-oleate, POE sorbitan keto-oleate, and POE sorbitan epoxy-stearate. Secondary oxidative products consist of various aldehydes, ketones, and carboxylic acids. However, none of these small molecules has been thoroughly quantitated through the PS oxidation process and correlated to PS oxidation quantitatively. See, for example, Bensaid F, et al. Int J Pharm. 2022; 615:121496; Doshi N, et al. Pharm Res. 2021; 38 (3): 531-48; Doyle Drbohlav L M, et al. PDA J Pharm Sci Technol. 2019; 73 (4): 320-30; Gopalrathnam G, et al. PDA J Pharm Sci Technol. 2018; 72 (2): 163-75; Mould R, et al. Pharm Res. 2023; 40 (8): 1965-76; Schmidt A, et al. J Pharm Sci. 2020; 109 (6): 1924-32; Borisov O V, et al. J Pharm Sci. 2015 March; 104 (3): 1005-18; Dahotre S, et al. J Pharm Biomed Anal. 2018; 157:201-7; Evers D H, et al. J Chromatogr B Analyt Technol Biomed Life Sci. 2020; 1157:122287; Kishore R S, et al. Pharm Res. 2011; 28 (5): 1194-210; and Hvattum E, et al. J Pharm Biomed Anal. 2012; 62:7-16.

The level of PS oxidation in protein drug products is usually negligible, likely due to protective effects of the protein. The most prominent cause for PS degradation in protein drug products is hydrolysis, which can be caused by trace level of enzymes that co-purify with the drug product. But in the absence of proteins, chemical hydrolysis of PS is negligible in formulations with pH 5-7 at 2-8° C. over 2 years.

Instead, PS oxidation is the major cause of PS degradation in formulations that are substantially free of proteins (and thus substantially free of enzymes such as lipases or esterases). As used herein “substantially free” means that the formulation excludes the particular component, e.g., enzymes such as lipases or esterases, to an amount of no more than 1 wt % of the formulation, such as no more than 0.1 wt %, or no more than 0.05 wt %, and even to a level that cannot be detected by chemical analysis, such as by liquid chromatography-mass spectrometry (LCMS). Placebos and diluents are among formulations substantially free of proteins. Without intending to be bound by theory, it is believed that PS can undergo oxidation facilitated by trace metals present in the raw material, bioreactor, container, and container closure system, and/or by peroxides from raw materials. Light exposure, buffer type and pH can also impact the rate and level of PS oxidation in formulations that are substantially free of proteins. Additionally, PS oxidation is related to particles observed in placebos and diluents. See, for example, Sutton A T, et al. Pharmaceuticals (Basel). 2024; 17 (2): 233; and Weber J, et al. Int J Pharm X. 2023; 6:100202.

There remains a need for methods for quantifying PS oxidation in formulations including placebo formulations.

PS80 oxidation has been characterized using LC-CAD where PS80 species eluting in the order POE, then POE monoesters, followed by POE higher order esters. See, for example, Borisov O V, et al. Anal Chem. 2011; 83 (10): 3934-42; Zheng X, et al. J Pharm Sci. 2023; 112 (3): 779-89; and Kranz W, et al. J Pharm Sci. 2020; 109 (10): 3064-77. However, all these POE related species can be further degraded into small molecules with extended incubation time or high oxidation stress; and therefore, general measurement of POE-related species cannot be used to quantitate PS80 oxidation. Furthermore, PS oxidation was described qualitatively using terms like mild, obvious, almost, or completely degraded.

Total PS content has been considered a critical quality attribute (CQA) for assessing PS stability in drug products. However, total PS content is ineffective for quantitation of PS80 oxidation, due to its lack of sensitivity and accuracy. While certain oxidative POE ester species have been proposed as indicators of PS oxidation, they also lack the sensitivity and accuracy to represent slight differences in PS80 oxidation stages.

Quantitative methods for monitoring the oxidation of polysorbates are described herein. The methods are useful for monitoring oxidative degradation of polysorbates such as PS80 in formulations, such as placebo or diluent formulations, in particular formulations that are substantially free of protein. The methods include determining the concentration of compounds that are markers of oxidation of polysorbates.

The methods described herein are fast, quantitative, and reliably indicate the degree of polysorbate oxidation, even for samples maintained under accelerated storage conditions such as increased temperature and/or oxidative stress.

Provided herein is a method for assessing oxidation of a polysorbate in a formulation, which includes determining a concentration of one or more oxidation markers of the polysorbate in a formulation that at least initially included the polysorbate, wherein the one or more oxidation markers include a carboxylic acid and/or an aldehyde.

The polysorbate can include one or more of PS20, PS40, PS60, or PS80. In some embodiments, the polysorbate is PS80.

The one or more oxidation markers can include one or more of acetic acid, formic acid, octanoic acid, or nonanoic acid. In some embodiments, the one or more oxidation markers includes octanoic acid. In some embodiments, the oxidation marker is octanoic acid.

When the polysorbate includes PS80, the one or more oxidation markers can include, or can be, octanoic acid.

In some aspects of the present disclosure, the formulation that at least initially included the polysorbate can be substantially free of protein.

The methods can include storing the formulation under storage conditions for a period of time prior to determining the concentration of one or more oxidation markers. Storage conditions can be characterized by conditions such as temperature (including constant or variable temperatures), exposure to light, exposure to air, exposure to oxidative stress (e.g., chemical oxidation by a chemical oxidant), and duration of storage.

The period of time prior to determining the concentration of one or more oxidation markers can be up to 61 months. In some embodiments, the period of time prior to determining the concentration of one or more oxidation markers can be, for example, less than one month, up to and including 1, 2, 3, 4, 5, 6, 7, 8, 9, 10, 11, or 12 months; up to 18 months; up to 24 months; up to 36 months; up to 48 months; up to 60 months; or up to 61 months and any range thereof such as between 1 to 36 months, 6 to 24 months, 12 to 18 months, etc.

The concentration of one or more oxidation markers can be determined repeatedly at various time points from the formulation that at least initially included the polysorbate. In this way, the progress of polysorbate oxidation can be monitored over time.

The storage conditions can include one or more stress conditions including thermal stress, oxidative stress, or a combination thereof. Thermal stress can include elevated temperature (e.g., a temperature higher than normal storage conditions, such as 5° C. or greater; 10° C. or greater; 15° C. or greater; 20° C. or greater; 25° C. or greater; 30° C. or greater; 35° C. or greater; or up to 40° C.). Thermal stress can also include temperature variation, such as temperature cycling. Temperature cycling can include repeated temperature variation in the range of, e.g., 2° C. and 40° C., or a narrower range, with the period of cycling in the range of, e.g., hourly, daily, weekly, monthly, or yearly.

In some embodiments, the storage conditions include storage at a constant temperature, wherein the constant temperature is between about 2° C. and about 40° C., and the period of time is up to about 61 months. Such periods of time can include less than one month, e.g., 1 to 30 days, 1 to 14 days or 21 days, etc. and up to and including 1, 2, 3, 4, 5, 6, 7, 8, 9, 10, 11, or 12 months; up to 18 months; up to 24 months; up to 36 months; up to 48 months; up to 60 months; or up to 61 months and any range thereof such as between 1 to 36 months, 6 to 24 months, 12 to 18 months, etc.

Determining the concentration of the one or more oxidation markers can include (a) derivatizing the one or more oxidation markers; and (b) detecting one or more of the derivatized oxidation markers by a liquid chromatography-mass spectrometry (LCMS) method.

Derivatizing the one or more oxidation markers which include a carboxylic acid and/or an aldehyde can include reacting the carboxylic acid and/or aldehyde with a hydrazine compound (e.g., 2-hydrazinoquinoline) to facilitate isolation and detection of the derivatized carboxylic acid and/or aldehyde.

Detecting one or more of the derivatized oxidation markers by a liquid chromatography-mass spectrometry (LCMS) method can include multiple reaction monitoring (MRM).

The formulation can include one or more of an amino acid, a sugar, and a buffer. In some cases, the amino acid can also act as a buffer. Suitable amino acids, sugars, and buffer are known in the art. In some embodiments, the formulation includes, e.g., histidine and/or sucrose, and has a pH of about 6.0.

In some cases, the formulation is a placebo formulation, selected to have a composition matching the composition of, e.g., a drug product or candidate drug product, except for the absence of an active ingredient. When the active ingredient in question is a protein (e.g., antibody), the placebo formulation can be substantially free of protein.

Also described herein are methods for assessing oxidation of polysorbate 80 (PS80) in a formulation, which includes determining a concentration of octanoic acid in the formulation at a first time point. The methods can further include determining a concentration of octanoic acid in the formulation at a second time point later than the first time point.

In some embodiments, determining the concentration of octanoic acid in the formulation includes (a) derivatizing the octanoic acid; and (b) detecting the derivatized octanoic acid by a liquid chromatography-mass spectrometry (LCMS) method. Derivatizing the octanoic acid can include derivatizing with 2-hydrazinoquinoline. The LCMS method can include multiple reaction monitoring (LC-MRM). The methods can further include storing the formulation under storage conditions for a period of time prior to determining the concentration of octanoic acid.

### EXAMPLES

The following examples are intended to further illustrate certain aspects of the subject technology and are not limiting in nature. Those skilled in the art will recognize, or be able to ascertain, using no more than routine experimentation, numerous equivalents to the specific substances and procedures described herein.

In the present study, PS80 oxidative species were monitored under a variety of oxidation stress and incubation lengths. Several secondary oxidative species were identified that continuously grew under extended oxidation conditions until all POE species and primary oxidative POE species disappeared. The levels of oxidative species under different oxidative stresses were also compared, and the most indicative products of PS80 oxidation (which showed higher concentrations with increased oxidative stress) were selected for further study. The detection limit of the selected oxidative products were further evaluated. Octanoic acid had a detection limit and dynamic range that allowed its concentration to indicate the degree of PS80 oxidation, at both initial and late stages. The concentration of octanoic acid was also independent of stress conditions.

### Materials and Methods

Acquity UPLC BEH 130 C4 column, 1.7 μm, 2.1 mm×50 mm (Part No. 186004496), Acquity UPLC BEH C18 column 1.7 μm, 2.1×50 mm (Part No. 186004660) and MAX column, 30 μm, 2.1 mm×20 mm (Part No. 186002052) were purchased from Waters (Milford, MA, USA). Super Refined Polysorbate 80 (SR PS80) was purchased from Croda (Princeton, NJ, USA). 2,2′-Dipyridyl disulfide (DPDS), triphenylphosphine (TPP), 2-hydrazinoquinoline (HQ), deuterated acetic acid (acetic acid-d4), FeCl₃ and peroxide were purchased from Sigma-Aldrich (St. Louis, MO, USA), LC-grade water and acetonitrile were purchased from Fisher Scientific (Houston, TX, USA).

Forced degradation of PS80 placebo samples

F1-F5 placebo samples were prepared by spiking 0 ppb, 1 ppb, 3 ppb, 10 ppb and 30 ppb FeCl₃, respectively, from 10 ppm FeCl₃ stock solution into a placebo formulation containing 10 mM histidine, 10% sucrose, 0.1% PS80 and pH 6.0. Each sample, 3 mL in volume, was aliquot to a 6R vial and transferred to a stability chamber. The samples were then incubated at 5° C., 25° C., or 40° C. for different lengths of time.

Placebo samples for mAb were prepared with different formulations based on the programs they were associated with and incubated at 5° C. for various durations. 0.1% PS80 placebo samples were prepared by incubating 0.1% SR PS80 in 10 mM histidine, pH 6.0 and incubated at 37° C. for up to 42 days. 0.1% PS80 stressed samples were prepared by incubating 0.1% SR PS80 in 0.02% H₂O₂ and 200 ppb FeCl₃ at 5° C. for up to 19 days.

Characterization of PS80 by liquid chromatography-charged aerosol detection (LC-CAD)

The characterization of PS80 in placebo samples was performed using an LC-CAD system. The reversed phase chromatography utilized an Acquity BEH C4 column (2.1 mm×50 mm, 1.7 mm) with mobile phase A as 0.1% formic acid in water and mobile phase B as 0.1% formic acid in acetonitrile. The gradient was initiated at 1% B, increased to 20% B in 5 mins and then gradually increased to 99% B in 35 mins. The gradient was held at 99% B for 5 mins and returned to 1% B for equilibration for another 5 mins. The flow rate was set at 0.1 mL/min and column temperature was maintained at 40° C. The experiment was conducted on a Thermo UltiMate 3000 instrument coupled with a Corona Ultra CAD detector with nitrogen pressure set at 80 psi.

Derivatization of ketone, aldehyde, and acid oxidation products for LC-MS Analysis

The derivatization reaction was performed by adding 2 μL of oxidized PS80 sample to an acetonitrile solution containing 1 mM DPDS, 1 mM TPP and 1 mM m (HQ). Acetic acid-d4 was added in the reaction solution as an internal standard. The reaction mixture was incubated at 60° C. for 30 min. Following incubation, the derivatives in the reaction mixture were injected to an LC-MS for data acquisition.

LC-MS method for small molecule analysis

A Waters Acquity I-class ultra-high performance liquid chromatography coupled with a Thermo Q Exactive mass spectrometer was used to identify derivatives of PS80 oxidation products. The separation was achieved using an Acquity BEH C18 column (2.1×50 mm, 1.7 μm) with a gradient of mobile phase of 0.05% aqueous acetic acid containing 2 mM ammonium acetate over a 10-min run. The gradient was set 10% B to 60% B in 4 min; and linear ramp to 95% B in 8 min; followed by a 2 min wash time with 100% B. The flow rate was 0.5 mL/min and column temperature was maintained at 40° C. The eluent was directed to the mass spectrometer in positive ESI mode to ionize compounds with spray voltage 3.5 kV, capillary temperature 270° C., Aux gas heater temperature 450° C. and S-lens RF level 50.

MRM quantitation of PS80 oxidation products Separation was performed on an Agilent ultra-performance liquid chromatography (UPLC) system (Agilent Technologies, Santa Clara, CA, USA) using the same gradient as described above. The UPLC was coupled to an Agilent 6475 triple quadrupole mass spectrometer (Agilent Technologies, Santa Clara, CA, USA) in positive ESI mode with spray voltage 3.5 kV, gas temperature 200° C., gas flow 12 L/min, sheath gas temperature 300° C., and sheath gas flow 11 L/min.

Post-acquisition data processing

Data were acquired using MassHunter Data Acquisition software (version 12.1). Afterwards, raw data was transferred to Skyline software (version 24.1) for quantitative analysis. Measurement of concentration was conducted by normalizing the peak area of the target ion with an internal standard. Concentrations were calculated using a standard curve generated by standards. Figures were generated by GraphPad software (version 10.1.316).

### Results

Characterization of PS80 oxidation with LC-CAD

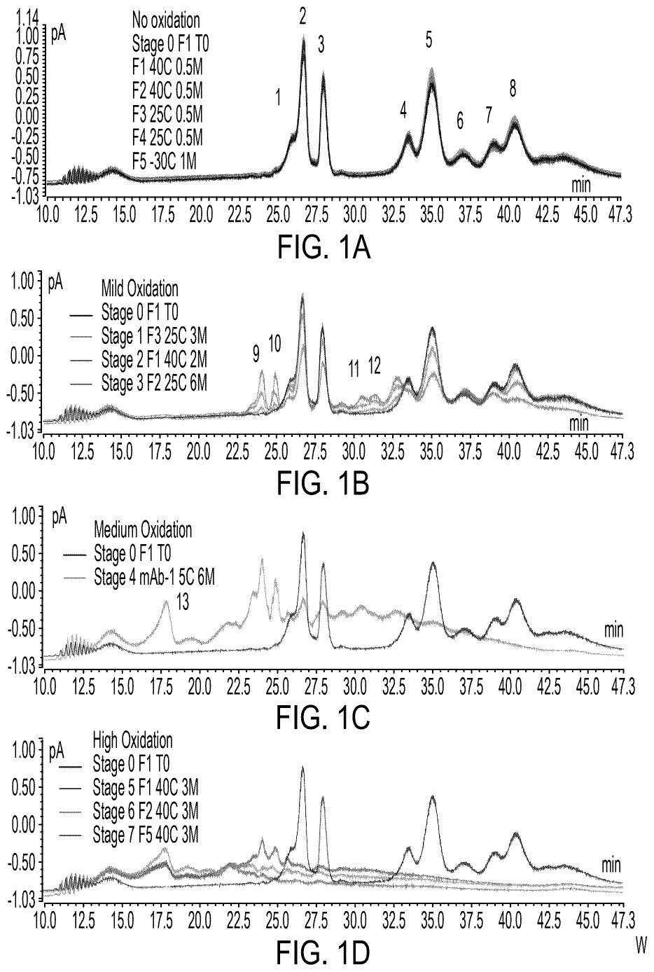

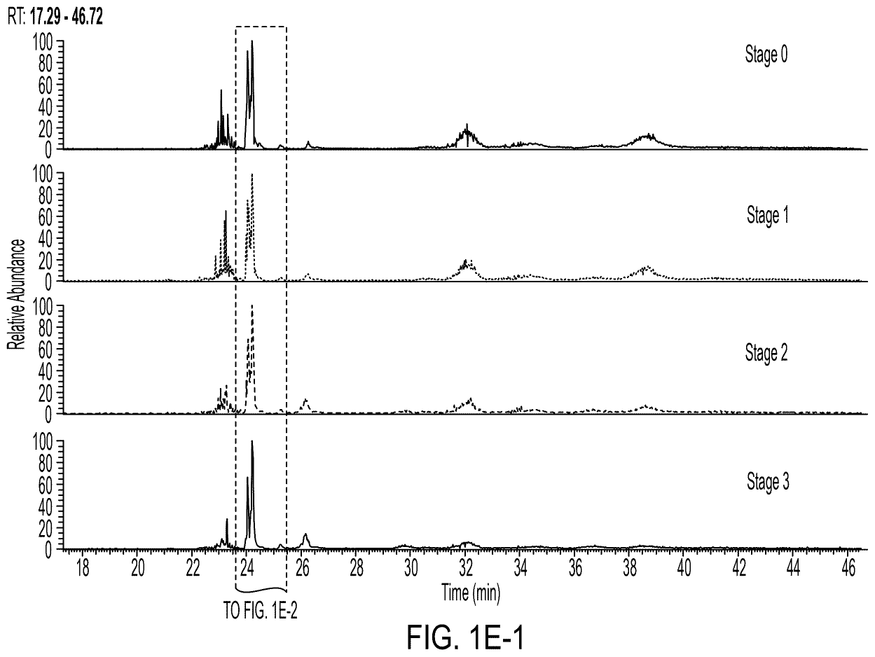

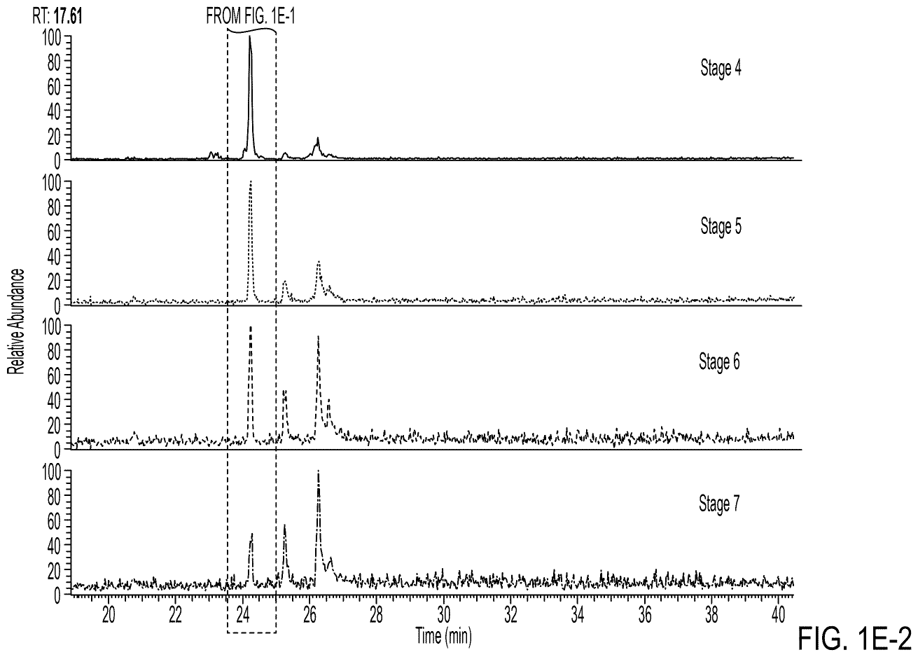

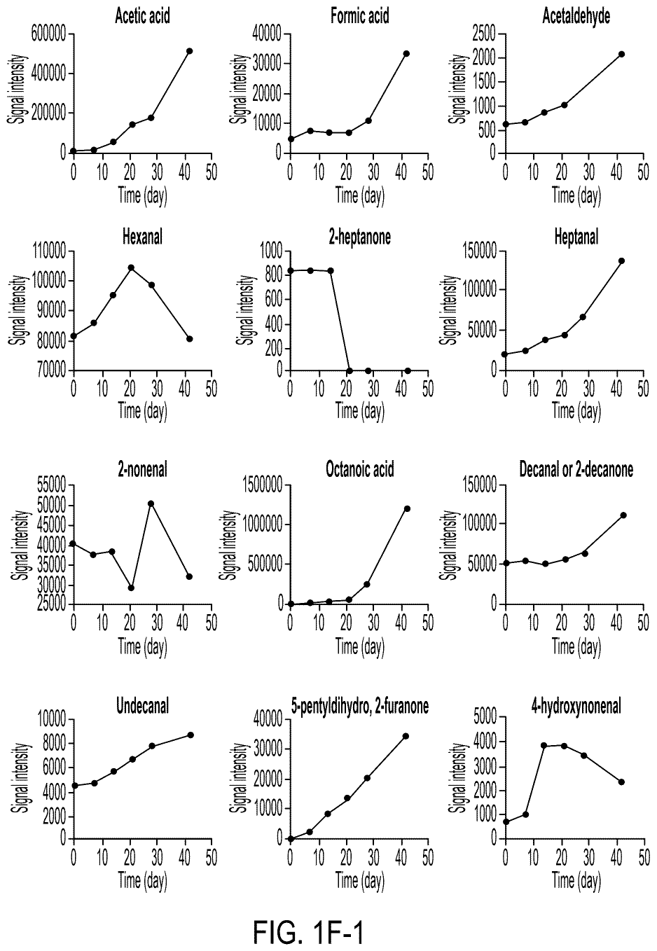

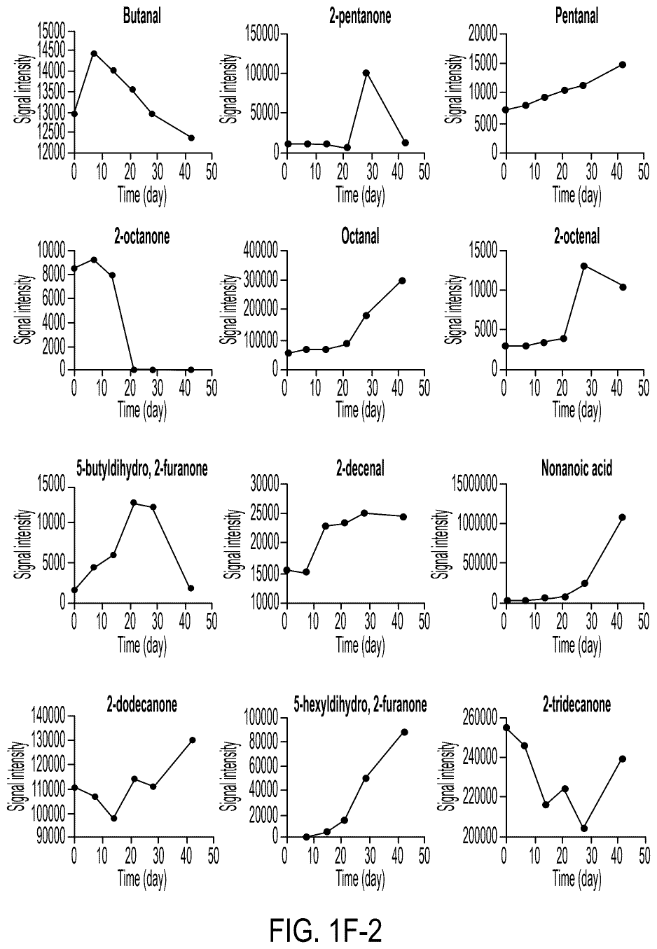

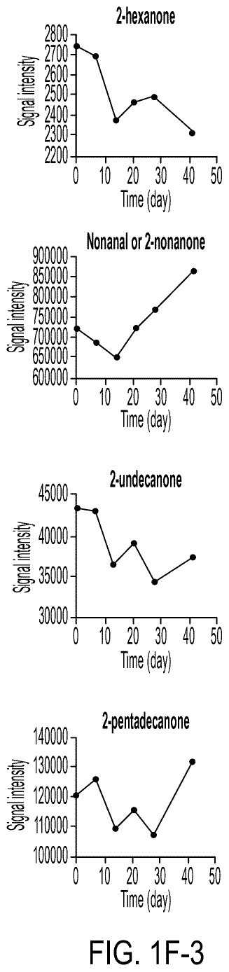

Oxidation of PS80 can be monitored by LC-CAD for placebo samples. PS80 and PS80 oxidative species can be separated using a reversed-phase column and detected by CAD, which are shown in FIGS. 1A-1D. All POE, POE monoester and POE high order ester species can be observed with peak 1-8 when there was no oxidation occurred as in FIG. 1A.

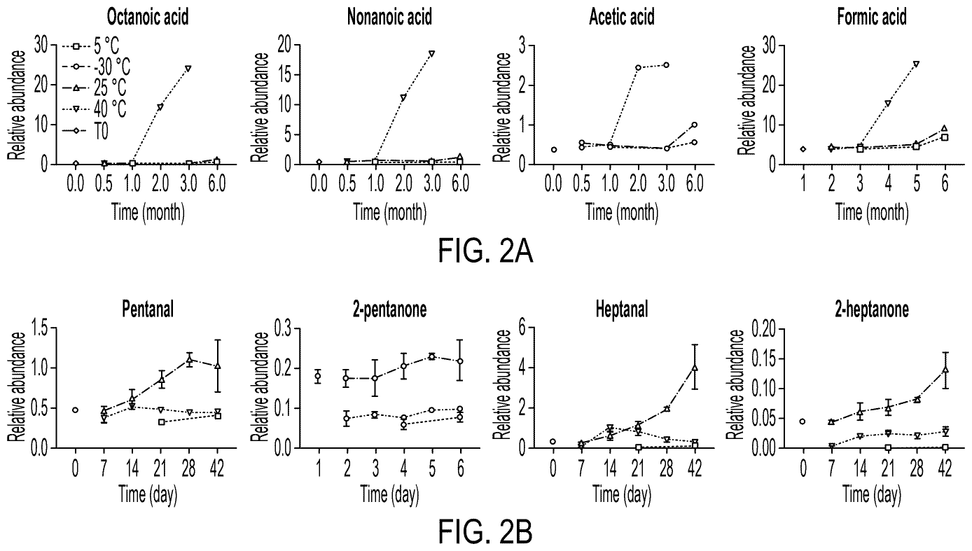

When oxidation began, during the mild oxidation stage, PS80 oxidative species begin to appear as shown in FIG. 1B. At this stage peaks 9-12 eluting from 22.5-25 mins and 29-33 mins were first observed, while peaks 1-8 exhibited a slight decrease in peak area. As oxidation progressed, peaks 9-12 continued to grow (FIG. 1B), and a new peak, identified as peak 13, eluting from 15-25 mins appeared in FIG. 1C. Meanwhile, peaks 1-8 dramatically decreased, marking the moderate oxidation stage. By the end of PS80 oxidation, defined as the high oxidation stage, all PS80 species and PS80 oxidative species (peaks 1-13) decreased and degraded into small molecules. Peak 2, which is the expected main species of PS80 gradually decreased throughout all stages, as shown in FIGS. 1E-1 to 1E-2. Quantitation of total PS80 content and POE sorbitan monooleate (EIC 309.27, eluting at 24 min) in each oxidation stage is presented in Table 1. The total PS80 content was quantitated using a calibration curve of PS80, while the remaining POE sorbitan monooleate levels were quantitated by comparing the POE sorbitan monooleate in a sample without oxidation. The level of PS80 oxidation was better defined by the decrease in POE sorbitan monooleate levels as its changes better reflecting the changes of PS80 profiles in FIG. 1A.

TABLE 1
Remaining POE sorbitan mono-oleate at different PS80
oxidation stage, eluting between 24-25 min; and
total PS80 content at different oxidation stage

| | Remained POE sorbitan | |
|---|---|---|
| | monolete (EIC 309.27) | Total PS80 content |
| Stage 0 | 100.00% | 0.089% |
| Stage 1 | 81.02% | 0.093% |
| Stage 2 | 78.74% | 0.094% |
| Stage 3 | 82.43% | 0.082% |
| Stage 4 | 16.48% | 0.072% |
| Stage 5 | 1.14% | 0.057% |
| Stage 6 | 0.55% | 0.045% |
| Stage 7 | 0.23% | 0.032% |

Based on the remaining POE sorbitan monooleate levels, PS80 oxidation was classified into 8 stages, from stage 0 to stage 7, spanning no oxidation to high oxidation. Although the remaining POE sorbitan monooleate levels were similar in mild oxidation stages 1-3, all POE species, including peaks 1-13, exhibited significantly different changes across these stages. Similarly, in the high oxidation stages 5-7, the differences between each stage were also indistinguishable based on the remaining levels of POE sorbitan monooleate, although the profiles of POE species exhibited some differences. Therefore, these stages were classified into three distinct stages rather than one. The inability to distinguish stages 1-3 and 5-7 by monooleate levels suggests that an alternative method is necessary to define these levels.

Detection and quantitation of potential PS80 oxidation products

PS80 oxidation was characterized by a decrease of POE and POE ester species, accompanied by an increase in unknown oxidative species, which are mostly likely aldehydes, ketones, and acids generated by fatty acid oxidation with breakage of the fatty acid chain. Therefore, quantitation of PS80 oxidation species is an alternative approach to quantitate PS80 oxidation. 0.1% (w/w) PS80 placebo formulation was incubated at 37° C. from 0 to 42 days to determine if its oxidation products accumulated over time. Major PS80 oxidation products including multiple aldehydes, ketones, and acids were identified and quantified (FIGS. 1F-1 to 1F-3). Among these PS80 oxidation products, acetic acid, acetaldehyde, formic acid, pentanal, heptanal, octanal, octanoic acid, decanal, nonanoic acid, 5-pentyldihydro-2-furanone, and 5-hexyldihydro-2-furanone increased during incubation, which were considered as potential markers to indicate PS80 oxidation levels.

Selection of marker to indicate PS80 oxidation stages

To determine if the potential oxidation markers accurately represented PS80 oxidation levels in various conditions, PS80 oxidation were tested under different temperatures, different oxidative stresses, and different incubation times. Potential markers showing temperature-dependent, oxidative stress-dependent, and time-dependent trends were quantified to select the most suitable marker. Acids such as acetic acid, formic acid, octanoic acid, and nonanoic acids were positively correlated with oxidative stress at elevated temperatures (FIG. 2A). In contrast, aldehyde markers like pentanal, 2-pentanone, heptanal and 2-heptanone increased more rapidly at 25° C. compared to 40° C. (FIG. 2B). Among the acid markers, octanoic acid and nonanoic acid had lower background signals, with octanoic acid exhibiting higher intensity compared to nonanoic acid, leading to its further evaluation.

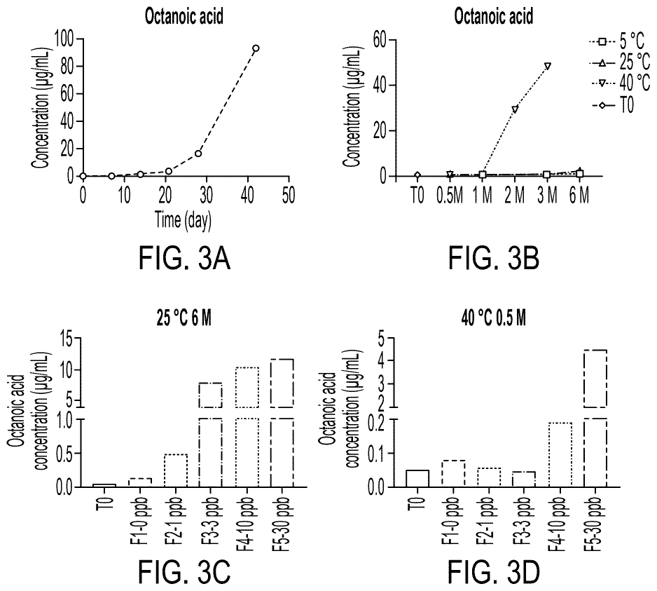

As illustrated in FIGS. 3A-3B, the concentration of octanoic acid continuously increased during incubation and was elevated at higher stressed temperatures compared to lower temperatures. Additionally, octanoic acid concentration showed a positive correlation with amount of iron spiked under all incubation conditions. FIGS. 3C-3D demonstrated that octanoic acid concentration reached its highest value with 30 ppb FeCl₃ spiked in at both 25° C. after 6 months of incubation and 40° C. after 0.5 months, compared to 0, 1, 3, 10 ppb FeCl₃ spiked in. Therefore, octanoic acid was considered suitable for quantifying PS80 oxidation.

Quantitation of octanoic acid with LC-MRM

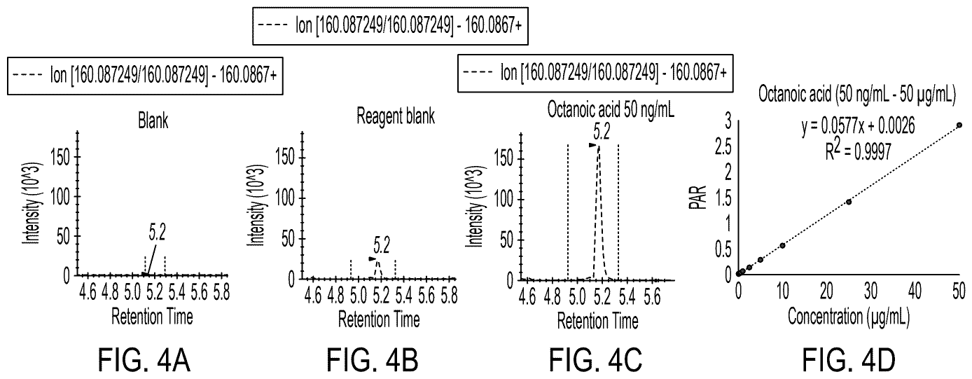

An LC-MRM method was developed to quantify octanoic acid of PS80 samples by using its precursor ion with m/z 286.19 and product ion m/z 160.09, with collision energy 20 eV. Transitions of octanoic acid were examined in blank, reagent blank, and octanoic acid standard. This transition state from 286.19 to 160.09 was absent in blank sample (FIG. 4A) but detectable in reagent blank (FIG. 4B). Consequently, the lower limit of quantification (LLOQ) was set to 50 ng/mL, which is 10 times of peak area of the reagent blank (FIG. 4C). The calibration curve, ranging from 50 ng/mL to 50 μg/mL, demonstrated good linearity with an R2 value of 0.9997 (FIG. 4D).

Correlation of PS80 oxidation stages with octanoic acid concentration

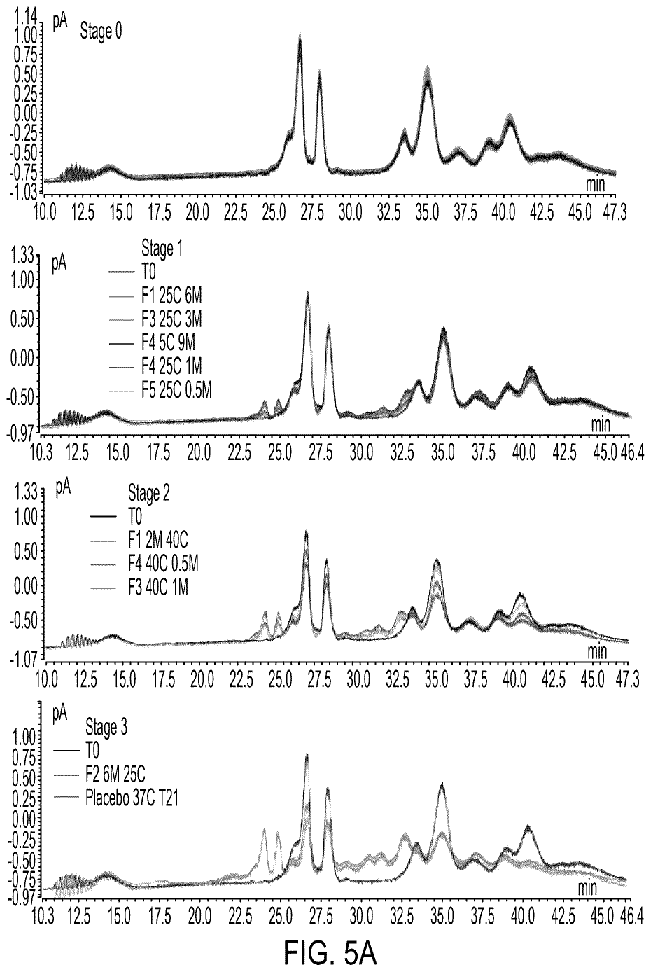

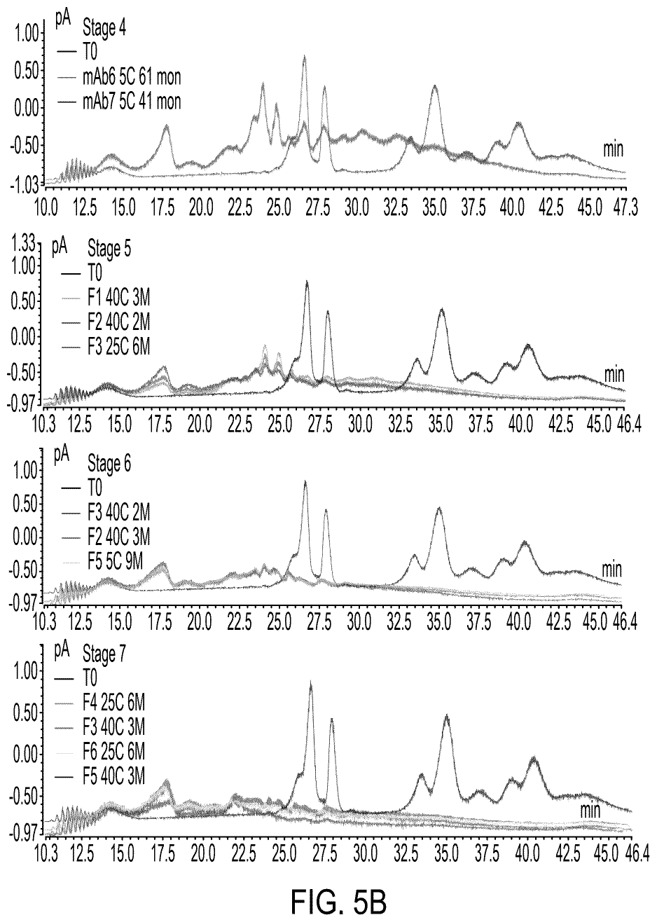

We overlaid the CAD chromatography of PS80 species under different oxidative stress and incubation time to define their PS80 oxidation stages, as seen in FIGS. 5A-B. For example, similar levels of PS80 oxidation were observed in samples F1 incubated at 40° C. for 2 months, F3 incubated at 40° C. for 1 month and F3 incubated at 40° C. for 0.5 month, which were classified as PS80 oxidation stage 2. A comparable level of PS80 oxidation was observed in the F2 sample incubated at 40° C. for 3 months, the F3 sample incubated at 40° C. for 2 months, and the F5 sample incubated at 5° C. for 9 months by overlaying the CAD spectra, indicating PS80 oxidation stage 6. The average concentration of octanoic acid measured for individual samples under various stressed condition and incubation times, but showing similar oxidation levels, was used to represent the octanoic acid level in each PS80 oxidation stage (Table 2).

TABLE 2
Octanoic acid concentration in stressed
samples from different oxidation stages.

| | |
|---|---|
| | Octanoic acid |
| Sample name | concentration (μg/mL) |

No oxidation
Stage 0

| | |
|---|---|
| F1 T0 | 0.05 |
| F1 40 C. 0.5M | 0.08 |
| F2 40 C. 0.5M | 0.06 |
| F3 23 C. 0.5M | 0.03 |
| F4 25 C. 0.5M | 0.08 |
| F5 −30 C. 1M | 0.08 |
| mAb1 placebo −80 C. | 0.03 |
| mAb2 placebo 5 C. 28 mon | 0.04 |
| mAb3 placebo 5 C. 26 mon | 0.04 |
| Avg | 0.05 |

Mild oxidation
Stage 1

| | |
|---|---|
| F4 25 C. 1M | 0.07 |
| FI 25 C. 6M | 0.14 |
| F3 25 C. 3M | 0.15 |
| F4 5 C. 9M | 0.18 |
| F5 25 C. 0.5M | 0.16 |
| mAb4 placebo 5 C. 39 mon | 0.16 |
| Avg | 0.16 |

Stage 2

| | |
|---|---|
| F4 40 C. 0.5M | 0.19 |
| F1 40 C. 2M | 0.23 |
| F3 40 C. 1M | 0.26 |
| 0.1% PS80 37 C. Day 14 | 0.21 |
| Ave | 0.22 |
| Std | 0.03 |
| CV % | 13.42% |

Stage 3

| | |
|---|---|
| F2 25 C. 6M | 0.49 |
| 0.1% PS80 37 C. Day 21 | 0.66 |
| mAb5 placebo 5 C. 25 mon | 0.47 |
| Avg | 0.54 |

Medium oxidation
Stage 4

| | |
|---|---|
| mAb6 placebo 5 C. 41 mon | 2.86 |
| mAb7 placebo 5 C. 61 mon | 2.84 |
| 0.1% PS80 37 C. Day 28 | 2.76 |
| Ave | 2.82 |

High oxidation
Stage 5

| | |
|---|---|
| F2 40 C. 2M | 5.49 |
| F1 40 C. 3M | 6.45 |
| F3 25 C. 6M | 7.76 |
| 0.1% PS80 37 C. Day 42 | 6.21 |
| Avg | 6.47 |

Stage 6

| | |
|---|---|
| F3 40 C. 2M | 9.39 |
| F2 40 C. 3M | 9.71 |
| F5 5 C. 9M | 9.89 |
| Avg | 9.66 |
| Std | 0.25 |
| CV % | 2.62% |

Stage 7

| | |
|---|---|
| F4 25 C. 6M | 10.39 |
| F3 40 C. 3M | 10.61 |
| F5 25 C. 6M | 11.72 |
| F5 40 C. 3M | 12.79 |
| Ave | 11.38 |

In Table 2, three sets of samples had been prepared in this stage. Set 1:0 ppb, 1 ppb, 3 ppb, 10 ppb and 30 ppb of iron had been spiked into 0.1% (w/w) 80, 10 mM histidine, 10% sucrose, pH 6.0 solution to generate F1, F2, F3, F4 and F5 samples. F1-F5 samples had been incubated at −30° C., 5° C., 25° C. and 40° C. for 0.5, 1, 3, 6 and 9 months. Set 2:0.1% (w/w) polysorbate 80 solution had been incubated under thermal stress condition at 37° C. for 7, 14, 21, 28 and 42 days. Set 3: mAb placebo samples were prepared along with each different program and incubated at 5° C. for different months. The CAD profile of PS80 species are shown in FIGS. 5A-5B.

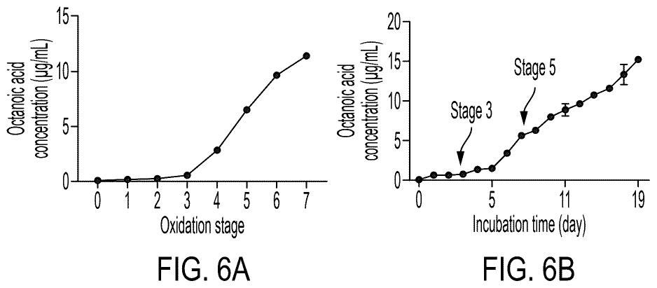

As illustrated in FIG. 6A, the average octanoic acid concentration in stage 0 was below 0.1 μg/mL, indicating no oxidation of PS80. During the mild oxidation stages (stage 1 to stage 3), 80% of POE sorbitan monooleate remained (Table 1), with gradual increase of octanoic acid to less than 1 μg/mL. Table 2 provides a more accurate and precise depiction of mild oxidation stages of PS80 through octanoic acid concentration. The decrease of POE sorbitan monolete was unable to demonstrate subtle differences during PS80 oxidation stage. However, octanoic acid concentration effectively classified mild oxidation stages with its concentration changes ranging from 0.16 to 0.66 μg/mL. Stages 1-3 were defined by octanoic acid levels of 0.1-0.2 μg/mL, 0.2-0.4 μg/mL and 0.4-0.7 μg/mL, respectively. Stage 4 can be defined as moderate oxidation stage, where an increase of multiple POE sorbitan/isosorbide oxidative species were observed along with a significant decrease of POE ester species. In this stage, octanoic acid concentration ranged between 1 to 5 μg/mL while remaining POE sorbitan monooleate ranged between 1% and 80%. Despite the broad range of stage 4, forced PS80 degradation placebo samples, F1-F5, failed to capture it. This stage was only captured with two other different sets of samples: 0.1% of PS80 incubated at 37° C. for 28 days, and mAb placebo samples incubated at 5° C. for 41 months and 61 months (Table 2).

PS80 oxidation progressed very rapidly starting from this stage despite the wide range of both octanoic acid and POE sorbitan monooleate, as indicated by steep slope in FIG. 6A. In order to better define this stage, we incubated 0.1% PS80 with 0.02% H₂O₂ and 200 ppb FeCl₃ at 5° C. for up to 19 days and measured the octanoic acid level each day. Stage 4 was captured at 3 data points from day 4 to day 6, with octanoic acid levels ranging from 1-5 μg/mL (FIG. 6B). Stage 4 was not further subdivided, as it progressed rapidly and the PS80 profile remained largely unchanged at this stage. When the oxidation stage exceeded stage 5, less than 1% of POE sorbitan monooleate remained in the sample, making it nearly impossible to quantify by CAD chromatogram (FIGS. 5A-5B). The oxidation levels can be precisely determined by measuring the concentration of octanoic acid produced during PS80 oxidation. The accuracy of the measurement increases at later stages, as more octanoic acid is generated. These stages, known as the high oxidation stages, are further divided into stage 5, stage 6, and stage 7 based on octanoic acid concentrations. Table 2 provides the octanoic acid concentrations for each of these stages, which range from 6-12 μg/mL.

Evaluation of octanoic acid as oxidation marker of PS80

Due to the high accuracy and precision of the results, measuring the concentration of octanoic acid in oxidized PS80 samples was considered a promising method to demonstrate the oxidation status of PS80.

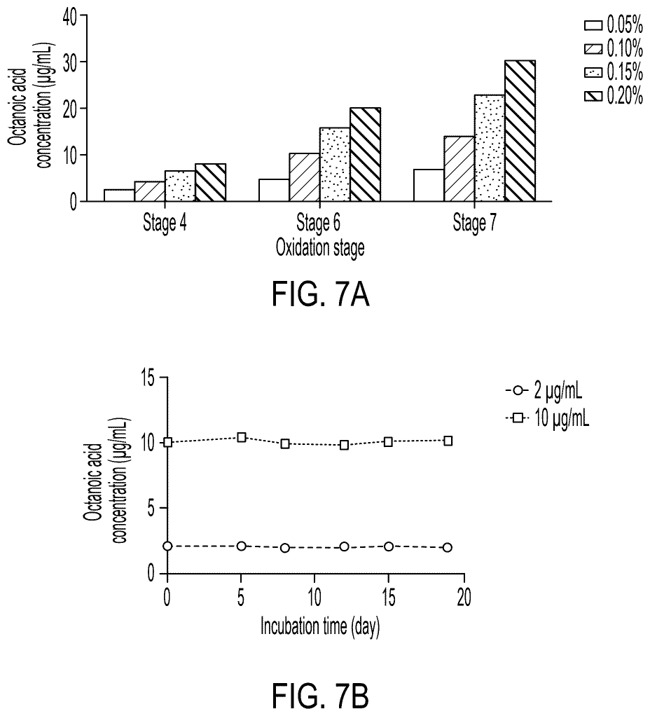

Firstly, to evaluate whether the production of octanoic acid is correlated with the percentage of PS80 in placebo samples, solutions containing 0.05%, 0.1%, 0.15% and 0.2% (w/w) PS80 were incubated with 0.02% H₂O₂ and 200 ppb FeCl₃ at 37° C. for up to 8 days, and octanoic acid concentration was measured (FIG. 7A). The results showed that octanoic acid concentration was positively correlated with PS80 percentage at all oxidation stage. Therefore, the corresponding octanoic acid concentration needs to be normalized to represent different oxidation stages if the placebo was formulated with varying concentrations of PS80.

Additionally, the stability of octanoic acid was evaluated by incubating it with 0.02% H₂O₂ and 200 ppb FeCl₃ at 37° C. for up to 19 days. The octanoic acid concentration remained unchanged during this period (FIG. 7B). Therefore, octanoic acid was confirmed as a stable marker even in later stages with severe oxidation conditions. The results demonstrated that octanoic acid can be used to determine the PS80 oxidation stage in formulation samples without further degradation under the tested oxidation conditions.

Only certain features and aspects of the subject technology and examples of its versatility are shown and described in the present disclosure. It is to be understood that the technology disclosed herein is capable of use in various other combinations and environments and is capable of changes or modifications. Thus, for example, those skilled in the art will recognize, or be able to ascertain, using no more than routine experimentation, numerous equivalents to the specific substances, procedures and arrangements described herein. Such equivalents are considered to be within the scope of the following claims.

What is claimed is:

1. A method for assessing oxidation of a polysorbate in a formulation, comprising determining a concentration of one or more oxidation markers of the polysorbate in a formulation that at least initially included the polysorbate, wherein the one or more oxidation markers comprise a carboxylic acid and/or an aldehyde.

2. The method of claim 1, wherein the polysorbate comprises polysorbate 80 (PS80).

3. The method of claim 1, wherein the one or more oxidation markers comprise acetic acid, formic acid, octanoic acid, or nonanoic acid.

4. The method of claim 1, wherein the one or more oxidation markers comprise octanoic acid.

5. The method of claim 1, wherein the formulation is substantially free of protein.

6. The method of claim 1, further comprising storing the formulation under storage conditions for a period of time prior to determining the concentration of one or more oxidation markers.

7. The method of claim 6, wherein the period of time is up to about 61 months.

8. The method of claim 6, wherein the storage conditions include one or more stress conditions comprising thermal stress, oxidative stress, or a combination thereof.

9. The method of claim 6, wherein the storage conditions include storage at a constant temperature, wherein the constant temperature is between about 2° C. and about 40° C., and the period of time is up to about 61 months.

10. The method of claim 1, wherein determining the concentration of the one or more oxidation markers includes:
(a) derivatizing the one or more oxidation markers; and
(b) detecting one or more of the derivatized oxidation markers by a liquid chromatography-mass spectrometry (LCMS) method.

11. A method for assessing oxidation of polysorbate 80 (PS80) in a formulation, comprising determining a concentration of octanoic acid in the formulation at a first time point.

12. The method of claim 11, further comprising determining a concentration of octanoic acid in the formulation at a second time point later than the first time point.

13. The method of claim 12, wherein determining the concentration of octanoic acid in the formulation includes:
(a) derivatizing the octanoic acid; and
(b) detecting the derivatized octanoic acid by a liquid chromatography-mass spectrometry (LCMS) method.

14. The method of claim 13, wherein derivatizing the octanoic acid includes derivatizing with 2-hydrazinoquinoline.

15. The method of claim 13, wherein the LCMS method includes multiple reaction monitoring (LC-MRM).

16. The method of claim 11, further comprising storing the formulation under storage conditions for a period of time prior to determining the concentration of octanoic acid.

17. The method of claim 16, wherein the period of time is up to about 61 months.

18. The method of claim 16, wherein the storage conditions include one or more stress conditions comprising thermal stress, oxidative stress, or a combination thereof.

19. The method of claim 16, wherein the storage conditions include storage at a constant temperature, wherein the constant temperature is between about 2° C. and about 40° C., and the period of time is up to about 61 months.

20. The method of claim 19, further comprising determining a concentration of octanoic acid in the formulation at a second time point later than the first time point.
